(19) United States
(12) Patent Application Publication
(10) Pub. No.: US 2022/0122141 A1
(43) Pub. Date: Apr. 21, 2022

(54) TRAIN ANY BUDDY REAL-TIME ONLINE MATCHING SERVICE

(71) Applicant: Rohit Bhardwaj, Fremont, CA (US)

(72) Inventor: Rohit Bhardwaj, Fremont, CA (US)

(21) Appl. No.: 17/076,001

(22) Filed: Oct. 21, 2020

(51) Int. Cl. G06Q 30/06; G06Q 10/10; G06Q 20/32; G06Q 20/06; G06Q 40/04; G06Q 20/38; G06Q 30/02; G06Q 50/00; G16H 20/30; H04L 29/06; G09B 19/00; G01C 21/34

(52) U.S. Cl. CPC G06Q 30/0611; G01S 19/51; G06Q 30/0625; G06Q 10/1095; G06Q 20/3267; G06Q 20/065; G06Q 40/04; G06Q 20/381; G06Q 30/0233; G06Q 30/0215; G06Q 30/0208; G06Q 50/01; G16H 20/30; H04L 63/0861; G09B 19/003; G01C 21/3438; G06Q 30/0639

(57) ABSTRACT

A system and method for providing an online, real-time exercise buddy and trainer matching service is disclosed. The system includes a trainer mobile device, an exercise buddy a trainer's current location data to the web server, a trainer query processor for responding to an exercise buddy query for available trainers, and a trainer messenger for communicating with the exercise buddy mobile devices. The exercise buddy mobile device has an exercise buddy trainer locator for obtaining identities and locations of one or more trainers currently available, an exercise buddy query generator for generating a trainer profile query to one of the currently available trainers, and an exercise buddy messenger for communicating with one or more of the trainer messengers. The web server has a trainer tracker for communicating with one or more of the trainer locators to maintain a set of currently available trainers and their respective current locations, a query manager for receiving and processing requests from one or more exercise buddy trainer locators, and a notification manager for providing all system notifications to the exercise buddy mobile device and trainer mobile device regarding appointments and messages.

### TECHNICAL FIELD

This application relates in general to a system and method for providing a fitness training aid, and more specifically, to a system and method for providing an online, on demand, and real-time trainer and exercise buddy matching service.

### BACKGROUND

There is a growing disconnect between home exercise buddies and certified exercise trainers. Exercise buddies are locating potential trainers and running due diligence before engaging a trainer. This has drastically reduced the value of the trainer's position in the transaction. Exercise buddies simply need to be shown a trainer profile for them to contact.

Therefore, a need exists for a system and method for providing an online, on demand, and real-time trainer and buddy matching service. The present invention attempts to address the limitations and deficiencies in the existing solutions according to the principles and embodiments disclosed herein to provide a missing link between eager trainers and exercise buddies.

### SUMMARY

In accordance with the present invention, the above and other problems are solved by providing a system and method for an online, on demand, real-time trainer and exercise buddy matching service according to the principles and example embodiments disclosed herein.

In one embodiment, the present invention is a system for providing an online, on demand, and real-time trainer and exercise buddy matching service. The system interconnects a trainer mobile device, one or more exercise buddy mobile devices, and a web server over the Internet. The web server includes a trainer tracker for maintaining location information for a trainer when logged into the system and indicating that he/she are available to meet with exercise buddies, a user manager for authenticating users via utilizing biometric data verification in the mobile applications as a form of authentication and multi-factor authentication, a query manager for connects an exercise buddy's query with a requested nearby trainer, an offer manager for receiving, maintaining, forwarding, and storing communication between buddies and trainers associated with offers from trainers and acceptance from buddies for training services, a notification manager 507 for providing notifications to exercise buddies and trainers for appointments, messages, and offers, and a user map and router for generating travel routes for exercise buddies and trainers to travel from a current location to a meeting location.

In another aspect of the present disclosure, the trainer mobile device and the buddy mobile device comprise smartphones, tablets, and laptop computers.

In another aspect of the present disclosure, the web server may be utilized for queries, offers, meeting scheduling, and user functions via a web browser.

In another aspect of the present disclosure, the user map and router obtains GPS location data from the mobile devices using built-in GPS receivers.

In another aspect of the present disclosure, the system utilizes in-app currency provided in in-app coins.

In another aspect of the present disclosure, the in-app coins may be traded by users.

In another aspect of the present disclosure, the in-app coins can be redeemed for cash each month.

In another aspect of the present disclosure, the trainer and training buddy may meet for training virtually using video-based communication.

In another aspect of the present disclosure, the query manager further performs searching for trainers having desired characteristics and services.

In another aspect of the present disclosure, the query manager shares training buddy queries with nearby trainers and generating trainer offers to training buddies.

In another embodiment, the present invention is a method for providing an online, on demand, and real-time trainer and exercise buddy matching service. The method defines trainer characteristics and services to be obtained, searches for available trainers matching the trainer characteristics and services, selects a first available trainer to solicit an offer, transmits a message to the first available trainer, receiving an offer for services, and accepting the offer for services.

In another aspect of the present disclosure, the offer for services is transmitted by the first available trainer.

In another aspect of the present disclosure, the offer for services is transmitted by a second available trainer before receiving a message from the training buddy.

In another aspect of the present disclosure, the method further obtains location data for available trainers and training buddies, utilizes the location data in searching, and generates a travel route presented with a map for the training buddies and trainers

These and other objectives of the present invention will become obvious to those of ordinary skill in the art after reading the following detailed description of the preferred embodiments. It is to be understood that the foregoing general description and the following detailed description are exemplary, and are intended to provide further explanation of the invention as claimed.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment of a system that provides an online, on demand, and real-time trainer and exercise buddy matching service according to the present invention.

FIG. 2a is a block diagram illustrating an exemplary hardware architecture of a computing device.

FIG. 2b is a block diagram illustrating an exemplary logical architecture for a client device.

FIG. 2c is a block diagram showing an exemplary architectural arrangement of clients, servers, and external services.

FIG. 2d is another block diagram illustrating an exemplary hardware architecture of a computing device.

FIG. 3 illustrates another example embodiment of a block diagram of a training buddy's mobile application within a system for an online, on demand, and real-time trainer and exercise buddy matching service according to the present invention.

FIG. 4 illustrates another example embodiment of a block diagram of a trainer's mobile application within a system for an online, on demand, and real-time trainer and exercise buddy matching service according to the present invention.

FIG. 5 illustrates another example embodiment of a block diagram of a web server within a system for an online, on demand, and real-time trainer and exercise buddy matching service according to the present invention.

FIG. 6 illustrates a flowchart corresponding to a method performed by software components providing a system for an online, on demand, and real-time trainer and exercise buddy matching service according to the present invention.

### DETAILED DESCRIPTION

This application relates in general to a system and method for providing a fitness training aid, and more specifically, to a system and method for an online, on demand, and real-time trainer and exercise buddy matching service according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly.”

The term “mobile application” refers to an application executing on a mobile device such as a smartphone, tablet, and/or web browser on any computing device.

The terms “trainer,” “client,” and “user” refer to an entity, e.g. a human, using the trainer-buddy matching mobile application including any software or smart device application(s) associated with the invention. The term user herein refers to one or more users.

The term “invention” or “present invention” refers to the invention being applied for via the patent application with the title “Train Any Buddy Real-Time Online Matching Service.” Invention may be used interchangeably with buddy system and exercise buddies.

The term “connection” refers to connecting any component as defined below by any means, including but not limited to, a wired connection(s) using any type of wire or cable for example, including but not limited to, coaxial cable(s), fiberoptic cable(s), and ethernet cable(s) or wireless connection(s) using any type of frequency/frequencies or radio wave(s). Some examples are including below in this application.

The terms “communicate” or “communication” refer to any component(s) connecting with any other component(s) in any combination for the purpose of the connected components to communicate and/or transfer data to and from any components and/or control any settings.

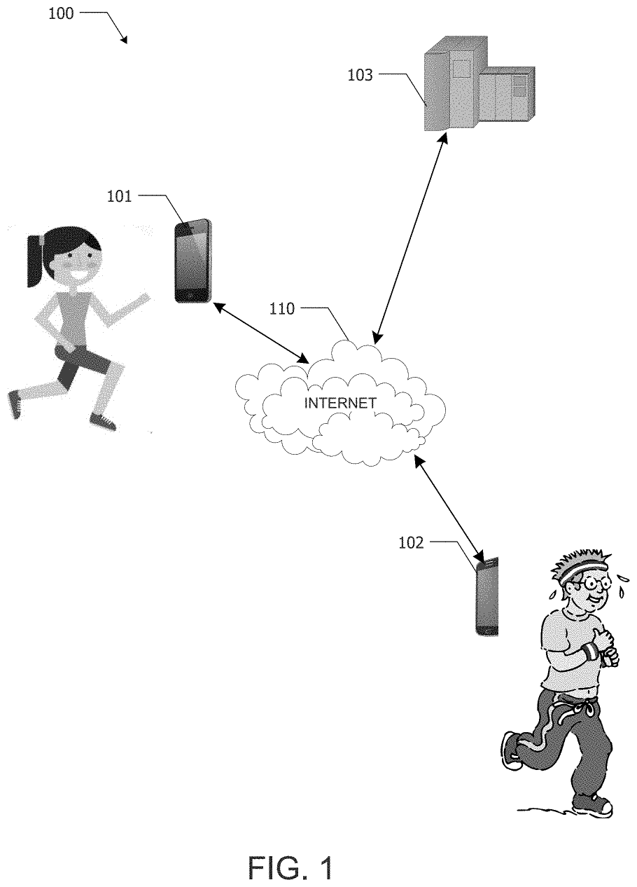

In general, the present disclosure relates to a system and method for providing a fitness training aid. To better understand the present invention, FIG. 1 illustrates an example embodiment for a system 100 for providing an online, on demand, and real-time trainer and buddy matching service according to the present invention. Exercise buddies would search for exercise partners or engage with a trainer. Once the exercise buddy had an interest in a trainer profile they would contact the trainer with hopes of engaging the trainer's services.

The system 100 attempts to change an outdated market and engagement cycle by allowing exercise buddies to search freely and when they are ready to engage services, these exercise buddies may contact or hire an on demand local trainer that indicates that he/she is available to meet at any particular moment in time. Using an exercise buddy's mobile application 102, the exercise buddies may search for available local trainers contact information 121-122. An available trainer has indicated that he/she is currently available using the trainer's mobile application 101 and the mobile application provides a current location to a system 100 web server 103.

Additionally, the system 100 also allows buddies to share the affordability of a trainer. A buddy may share their respective affordability/price they could afford and trainers who are interested could bid back. Typically, the system 100 allows 2 biddings (back and forth) and then OBO. This is a unique way to lower the burgeoning personal training costs.

Trainers may also reach out to the buddies as the system acts both ways. For the first time in the industry, even trainers will have visibility into who all want training. So far in existing systems, only the people who needed training used to reach out to trainers but now even trainers could reach out to potential buddies. The app has a hand raise feature i.e. who so ever needs training help, he/she raises hand and the trainers could see that request.

The system provides for group trainings. The App provides a feature for buddies to form groups and workout either online or in person. The system may utilizes online training using a video chatting and conference application such as Zoom™, Skype, and FaceTime™, when providing service Also the group members could invite a trainer-on demand to their Online or In person training class.

The system also uses its own in-app coins. Using the in-app currency, users are permitted to leveraged between buddies to train each other and earn coins. The coins could be redeemed for cash after end of each month. This is unique way of motivating people to earn quick gigs by training each other. As noted above, all of the above options are available both in-person and online training using a video chatting and conference application such as Zoom™ Skype, and FaceTime™.

Because the mobile application 101-102 typically executes on a smartphone or similar mobile device, these mobile applications may obtain an accurate current position as most smartphones contain a GPS receiver that is capable of sharing location data with any application on the device. When an exercise buddy uses his/her mobile application 102 to search for an available trainer, the mobile application 102 may obtain from the web server 103 identities of any trainer currently indicating availability using the trainer mobile application 101. The exercise buddies may choose a trainer who is closest to their current location.

Use of the system 100 permits exercise buddies to locate an available trainer when they are ready to engage a trainer while permitting the trainers to engage in other activities in their area while they are still available to meet exercise buddies 111. When a trainer meets with an exercise buddy, he/she may change his/her status to busy, for example, using the trainer mobile application 101. Such a system 100, provides instant gratification to both parties and allows both of them to meet when it is convenient without a lot of advance planning.

The invention may use any type of network such as a single network, multiple networks of a same type, or multiple networks of different types which may include one or more of a direct connection between devices, including but not limited to a local area network (LAN), a wide area network (WAN) (for example, the Internet), a metropolitan area network (MAN), a wireless network (for example, a general packet radio service (GPRS) network), a long term evolution (LTE) network, a telephone network (for example, a Public Switched Telephone Network or a cellular network), a subset of the Internet, an ad hoc network, a fiber optic network (for example, a fiber optic service (often known as FiOS) network), or any combination of the above networks.

Smart devices mentioned herein the present application may also use one or more sensors to receive or send signals; wireless signals such as Bluetooth™, wireless fidelity, infrared, Wi-Fi, or LTE. Any smart device mentioned in this application may be connected to any other component or smart device via wired communications (e.g., conductive wire, coaxial cable, fiber optic cable, ethernet cable, twisted pair cable, transmission line, waveguide, etc.), or a combination of wired and wireless communications. The invention's method and/or system may use a single server device or a collection of multiple server devices and/or computer systems.

The systems and methods described above, may be implemented in many different forms of applications, software, firmware, and hardware. The actual software or smart device application codes or specialized control software, hardware or smart device application(s) used to implement the invention's systems and methods is not limiting of the implementation. Thus, the operation and behavior of the systems and methods were described without reference to the specific software or firmware code. Software, smart device application(s), firmware, and control hardware can be designed to implement the systems and methods based on the description herein.

This new invention also has the ability to manage, control or communicate with multiple exercise buddies and trainers from one or more server or computer system locations without multiple interventions.

While all of the above functions are described to be provided to users via a mobile application on a smartphone, one of ordinary skill will recognize that any computing device including tablets, laptops, and general purpose computing devices may be used as well. In at least one embodiment, all of the services described herein are provided using web pages being accessed from the web server 201 using a web browser such as Safari™, Firefox™, Chrome™ DuckDuckGo™, and the like. All of the screen examples described herein show user interface elements that provide the functionality of the present invention. The arrangement, organization, presentation, and use of particular user input/output (I/O) elements including hyperlinks, buttons, text fields, scrolling lists, and similar I/O elements are shown herein for example embodiments only to more easily convey the features of the present invention. The scope of the present invention should not be interpreted as being limited by any of these elements unless expressly recited within the attached claims.

For the purposes of the example embodiment of FIG. 1, various functions are shown to be performed on different programmable computing devices that communicate with each other over the Internet 105. These computing devices may include smartphones 101a, laptop computers 101b, tablets (not shown), and similar devices so long as the disclosed functionality of the mobile application described herein is supported by the particular computing device. One of ordinary skill will recognize that this functionality is grouped as shown in the embodiment for clarity of description. Two or more of the processing functions may be combined onto a single processing machine. Additionally, it may be possible to move a subset of processing from one of the processing systems shown here and retain the functionality of the present invention. The attached claims recite any required combination of functionality onto a single machine, if required, and all example embodiments are for descriptive purposes.

For all of the above devices that are in communication with each other, some or all of them need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects, and in order to more fully illustrate one or more aspects. Similarly, although process steps, method steps, algorithms or the like may be described in a sequential order, such processes, methods, and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method or algorithm is carried out or executed. Some steps may be omitted in some aspect or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the aspects disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific aspects, at least some of the features or functionalities of the various aspects disclosed herein may be implemented on one or more general-purpose computers associated with one or more networks, such as for example, an end-user computer system, a client computer, a network server or other server system, a mobile computing device (e.g., tablet computing device, mobile phone, smartphone, laptop or other appropriate computing device), a consumer electronic device, a music player or any other suitable electronic device, router, switch or other suitable device, or any combination thereof. In at least some aspects, at least some of the features or functionalities of the various aspects disclosed herein may be implemented in one or more virtualized computing environments (e.g., network computing clouds, virtual machines hosted on one or more physical computing machines or other appropriate virtual environments).

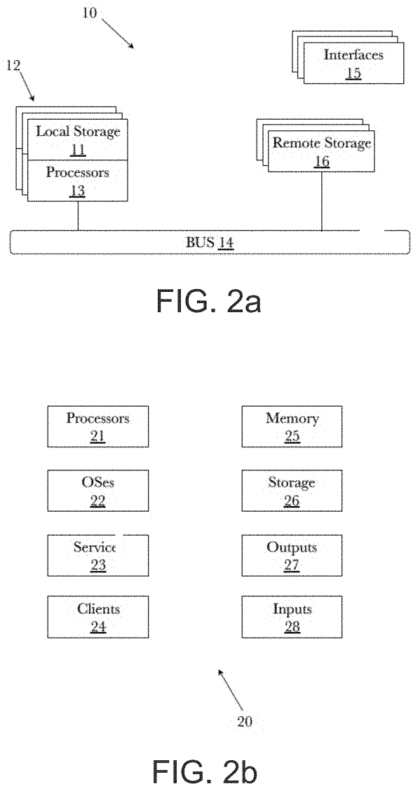

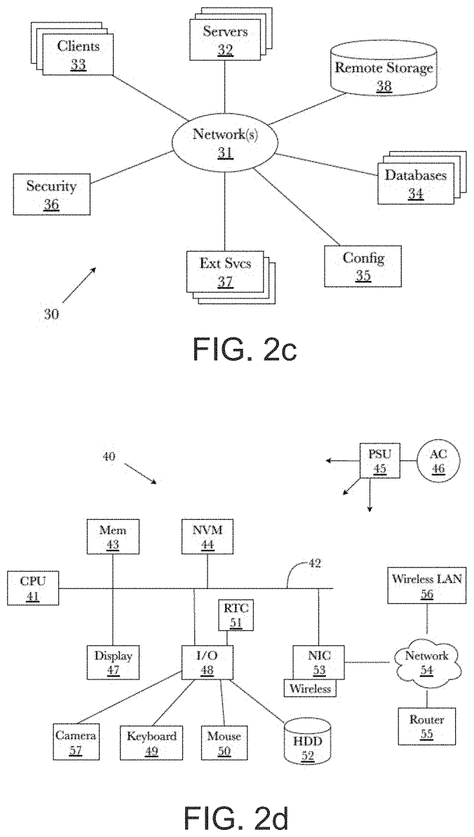

Referring now to FIG. 2a, there is a block diagram depicting an exemplary computing device 10 suitable for implementing at least a portion of the features or functionalities disclosed herein. The computing device 10 may be, for example, any one of the computing machines listed in the previous paragraph, or indeed any other electronic device capable of executing software- or hardware-based instructions according to one or more programs stored in memory. The computing device 10 may be configured to communicate with a plurality of other computing devices, such as clients or servers, over communications networks such as a wide area network, a metropolitan area network, a local area network, a wireless network, the Internet or any other network, using known protocols for such communication, whether wireless or wired.

In one aspect, the computing device 10 includes one or more central processing units (CPUs) 12, one or more interfaces 15, and one or more buses 14 (such as a peripheral component interconnect (PCI) bus). When acting under the control of appropriate software or firmware, a CPU 12 may be responsible for implementing specific functions associated with the functions of a specifically configured computing device or machine. For example, in at least one aspect, a computing device 10 may be configured or designed to function as a server system utilizing a CPU 12, local memory 11 and/or remote memory 16, and interface(s) 15. In at least one aspect, a CPU 12 may be caused to perform one or more of the different types of functions and/or operations under the control of software modules or components, which for example, may include an operating system and any appropriate applications software, drivers, and the like.

A CPU 12 may include one or more processors 13 such as for example, a processor from one of the Intel, ARM, Qualcomm, and AMD families of microprocessors. In some aspect, processors 13 may include specially designed hardware such as application-specific integrated circuits (ASICs), electrically erasable programmable read-only memories (EEPROMs), field-programmable gate arrays (FPGAs), and so forth, for controlling operations of a computing device 10. In a particular aspect, a local memory 11 (such as non-volatile random access memory (RAM) and/or read-only memory (ROM), including for example, one or more levels of cached memory) may also form part of a CPU 12. However, there are many different ways in which memory may be coupled to a system 10. Memory 11 may be used for a variety of purposes such as, for example, caching and/or storing data, programming instructions, and the like. It should be further appreciated that a CPU 12 may be one of a variety of system-on-a-chip-(SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may, for example, support other peripherals used with a computing device 10. Among the interfaces that may be provided are ethernet interfaces, frame relay interfaces, cable interfaces, DSL interfaces, token ring interfaces, graphics interfaces, and the like. In addition, various types of interfaces may be provided such as, for example, universal serial bus (USB), serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast ethernet interfaces, gigabit ethernet interfaces, serial ATA (SATA) or external SATA (ESATA) interfaces, high-definition multimedia interfaces (HDMI), digital visual interfaces (DVI), analog or digital audio interfaces, asynchronous transfer mode (ATM) interfaces, high-speed serial interfaces (HSSI), point of sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 2a illustrates one specific architecture for a computing device 10 for implementing one or more of the aspects described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, a single processor 13 handles communications as well as routing computations, while in other aspects a separate dedicated communications processor may be provided. In various aspects, different types of features or functionalities may be implemented in a system according to the aspect that includes a client device (such as a tablet device or smartphone running client software) and a server system (such as a server system described in more detail below).

Regardless of network device configuration, the system of an aspect may employ one or more memories or memory modules (for example, remote memory block 16 and local memory 11) configured to store data, program instructions for the general-purpose network operations or other information relating to the functionality of the aspects described herein (or any combinations of the above). Program instructions may control execution of or comprise an operating system and/or one or more applications, for example. Memory 16 or memories 11, 16 may also be configured to store data structures, configuration data, encryption data, historical system operations information or any other specific or generic non-program information described herein.

Because such information and program instructions may be employed to implement one or more systems or methods described herein, at least some network device aspects may include non-transitory machine-readable storage media, which, for example, may be configured or designed to store program instructions, state information, and the like for performing various operations described herein. Examples of such non-transitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device) or they may be removable such as swappable flash memory modules (such as “thumb drives” or other removable media designed for rapidly exchanging physical storage devices), “hot-swappable” hard disk drives or solid state drives, removable optical storage disks, or other such removable media, and that such integral and removable storage media may be utilized interchangeably. Examples of program instructions include both object code, such as may be produced by a compiler, machine code, such as may be produced by an assembler or a linker, byte code, such as may be generated by for example by a JAVA™ compiler and may be executed using a JAVA™ virtual machine or equivalent, or files containing higher level code that may be executed by the computer using an interpreter (for example, scripts written in Python™, Perl™, Ruby™, Groovy™, or any other scripting language).

In some aspects, systems may be implemented on a standalone computing system. Referring now to FIG. 2b, there is a block diagram depicting a typical exemplary architecture of one or more aspects or components thereof on a standalone computing system. A computing device 20 includes processors 21 that may run software that carry out one or more functions or applications of aspects, such as for example a client application 24. Processors 21 may carry out computing instructions under control of an operating system 22 such as, for example, a version of MICROSOFT WINDOWS™ operating system, APPLE macOS™ or iOS™ operating systems, some variety of the LINUX™ operating system, ANDROID™ operating system, or the like. In many cases, one or more shared services 23 may be operable in system 20, and may be useful for providing common services to client applications 24. Services 23 may, for example, be WINDOWS™ services, user-space common services in a LINUX™ environment or any other type of common service architecture used with an operating system 21. Input devices 28 may be of any type suitable for receiving user input including, for example, a keyboard, touchscreen, microphone (for example, for voice input), mouse, touchpad, trackball or any combination thereof. Output devices 27 may be of any type suitable for providing output to one or more users, whether remote or local to system 20, and may include, for example, one or more screens for visual output, speakers, printers or any combination thereof. Memory 25 may be RAM having any structure and architecture known in the art for use by processors 21, for example to run software. Storage devices 26 may be any magnetic, optical, mechanical, memristor or electrical storage device for storage of data in digital form (such as those described above, referring to FIG. 2a). Examples of storage devices 26 include flash memory, magnetic hard drive, CD-ROM, and the like.

In some aspects, systems may be implemented on a distributed computing network, such as one having any number of clients and/or servers. Referring now to FIG. 2c, there is a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. B. In addition, any number of servers 32 may be provided for handling requests received from one or more clients 33. Clients 33 and servers 32 may communicate with one another via one or more electronic networks 31, which may be in various aspects any Internet, wide area network, mobile telephony network (such as CDMA or GSM cellular networks), wireless network (such as WiFi, WiMAX, LTE, and so forth) or local area network (or indeed any network topology known in the art; the aspect does not prefer any one network topology over another). Networks 31 may be implemented using any known network protocols, including, for example, wired and/or wireless protocols.

In addition, in some aspects, servers 32 may call external services 37 when needed to obtain additional information, or to refer to additional data concerning a particular call. Communications with external services 37 may take place, for example, via one or more networks 31. In various aspects, external services 37 may comprise web-enabled services or functionality related to or installed on the hardware device itself. For example, in one aspect where client applications 24 are implemented on a smartphone or other electronic device, client applications 24 may obtain information stored on a server system 32 in the Cloud or on an external service 37 deployed on one or more of a particular enterprise's or user's premises. In addition to local storage on servers 32, remote storage 38 may be accessible through the network(s) 31.

In some aspects, clients 33 or servers 32 (or both) may make use of one or more specialized services or appliances that may be deployed locally or remotely across one or more networks 31. For example, one or more databases 34 in either local or remote storage 38 may be used or referred to by one or more aspects. It should be understood by one having ordinary skill in the art that databases in storage 34 may be arranged in a wide variety of architectures and use a wide variety of data access and manipulation means. For example, in various aspects one or more databases in storage 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some aspects, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system or a logical database within an overall database management system. Unless a specific meaning is specified for a given use of the term “database,” it should be construed to mean any of these senses of the word, all of which are understood as a plain meaning of the term “database” by those having ordinary skill in the art.

Similarly, some aspects may make use of one or more security systems 36 and configuration systems 35. Security and configuration management are common information technology (IT) and web functions, and some amount of each are generally associated with any IT or web system. It should be understood by one having ordinary skill in the art that any configuration or security subsystems known in the art now or in the future may be used in conjunction with aspects without limitation, unless a specific security 36 or configuration system 35 or approach is required by the description of any specific aspect.

FIG. 2d shows an exemplary overview of a computer system 40 as may be used in any of the various locations throughout the system. It is exemplary of any computer that may execute code to process data. Various modifications and changes may be made to a computer system 40 without departing from the broader scope of the system and method disclosed herein. A CPU 41 is connected to bus 42, to which bus is also connected to memory 43, nonvolatile memory 44, display 47, I/O unit 48, and network interface card (NIC) 53. An I/O unit 48 may, typically, be connected to peripherals such as a keyboard 49, pointing device 50, hard disk 52, real-time clock 51, camera 57, and other peripheral devices. A NIC 53 connects to a network 54, which may be the Internet or a local network, which local network may or may not have connections to the Internet. The system may be connected to other computing devices through the network via a router 55, wireless local area network 56 or any other network connection. Also shown as part of a system 40 is a power supply unit 45 connected, in this example, to a main alternating current (AC) supply 46. Not shown are batteries that could be present and many other devices and modifications that are well known, but are not applicable to, the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles or other integrated hardware devices).

In various aspects, functionality for implementing systems or methods of various aspects may be distributed among any number of client and/or server components. For example, various software modules may be implemented for performing various functions in connection with the system of any particular aspect, and such modules may be implemented to run on server and/or client components.

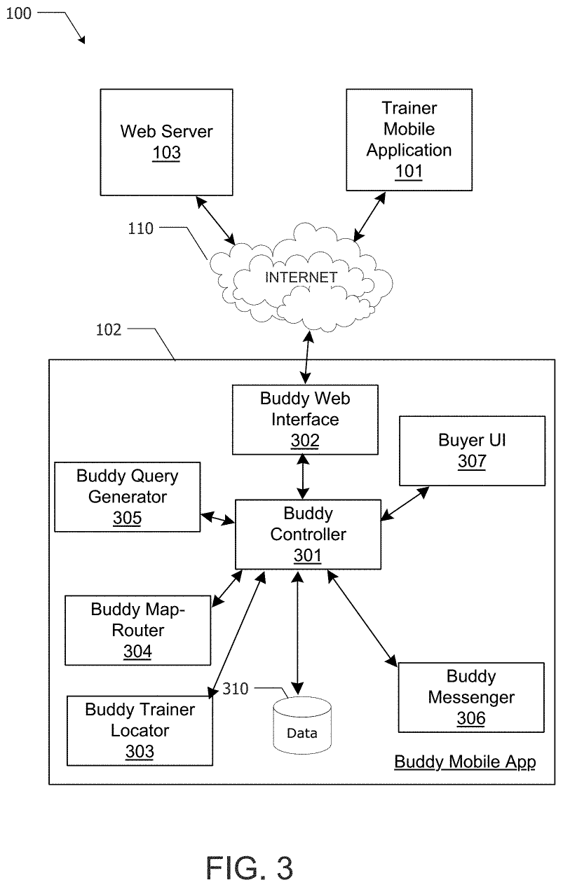

FIG. 3 illustrates another example embodiment of a block diagram of an exercise buddy mobile application 102 within a system for an online, real-time exercise buddy and trainer matching service according to the present invention. The exercise buddy mobile application 102 communicates with the web server 103 and a trainer mobile application 101 over the Internet 110. The exercise buddy mobile application 102 comprises a set of processing components to perform the processing described herein. The exercise buddy set of processing components includes an exercise buddy controller 301, an exercise buddy web interface 302, an exercise buddy trainer profile locator 303, an exercise buddy map router 304, an exercise buddy query generator 305, an exercise buddy messenger 306, and an exercise buddy UI 307.

The exercise buddy controller 301 generates and sends available trainer queries via the exercise buddy web interface 302 when requested by an exercise buddy using the exercise buddy mobile application 102 through the exercise buddy UI 307. The exercise buddy controller 301 also obtains current GPS location data from the exercise buddy's smartphone for use in providing location data to the web server 103 and the trainer mobile application 101. The exercise buddy controller 301 interacts with the remaining exercise buddy set of processing components to generate messages and data sent to one or more trainer mobile applications 101 and the web server 103 as needed. The exercise buddy controller 301 stores its data for use when operating into a local exercise buddy datastore 310 as needed.

The exercise buddy web interface 302 connects the exercise buddy mobile application 102 to the Internet 110 to send and receive communication from one or more trainer mobile applications 101 and the web server 103. The exercise buddy web interface 302 performs all necessary data formatting, data packet creation, data encryption for security, and data transmission and reception when the exercise buddy mobile application 102 communicates with other processing systems disclosed herein. The exercise buddy web interface 302 also is responsible for ensuring reception of any communications to other computing systems, the exercise buddy user interface (UI) 307, and for logging any errors or attempts to hack into the local exercise buddy data store 310. The exercise buddy controller 301 stores its data for use when operating into a local exercise buddy datastore 310 as needed.

The exercise buddy trainer locator 303 searches current available trainer data on the web server to locate an available trainer near the exercise buddy's current location. The exercise buddy trainer locator 303 obtains current GPS location data from a GPS receiver present in the mobile devices and may also determine the exercise buddy's current location using Wifi hot spot data and smartphone cell tower data when GPS data is unavailable. The exercise buddy trainer locator 303 presents the search results to the exercise buddy via the exercise buddy UI 307. The exercise buddy may select an available trainer from these search results and pass the available trainer data to the exercise buddy query generator 305 to contact the trainer. The exercise buddy trainer profile locator 303 stores its operating data for use when operating into a local exercise buddy datastore 310 as needed.

The exercise buddy map router 304 generates a route to a desired trainer's location from the exercise buddy's current location. The exercise buddy map router 304 obtains current GPS location data from a GPS receiver present in the mobile devices and may also determine the exercise buddy's current location using Wifi hot spot data, and smartphone cell tower data when GPS data is unavailable. The exercise buddy map router 304 may use map and location data from the web server 103 or other online map and route travel service such as Apple Maps™, Google Maps™, and similar web services that provide application programming interfaces (APIs) for use by applications on mobile devices. A route generated by the exercise buddy map router 304 may be optimized to generate a route having the lowest travel time, the lowest tolls, and the lowest amount of congestion from accidents, construction and the like. The exercise buddy map router 304 stores saved addresses and routes on available trainers and related operating data for use when operating into a local exercise buddy datastore 310 as needed.

The exercise buddy query generator 305 generates an exercise buddy query requesting an available trainer as identified in data obtained by the exercise buddy trainer locator 303 and transmits the exercise buddy query to the trainer mobile application 101. The exercise buddy query generator 305 receives all responses to these exercise buddy queries and presents them to the exercise buddy via the exercise buddy UI 307. The exercise buddy query generator 305 stores generated queries, the corresponding responses, and related operating data for use when operating into a local exercise buddy datastore 310 as needed.

The exercise buddy trainer profile searcher 306 locates trainers currently available in a geographic area near the exercise buddy's current location. The exercise buddy trainer searcher 306 obtains current GPS location data from a GPS receiver present in the mobile devices. The exercise buddy locator 303 may maintain a last known location for use when GPS data is unavailable and may also determine a current location using Wifi hot spot data and smartphone cell tower data when GPS data is unavailable. The exercise buddy trainer profile searcher 306 contacts the web server to obtain the identity and address of available trainers from the web server 103. Results of these searches are provided to the exercise buddy via the exercise buddy UI 307 so that the exercise buddy may attempt to obtain a trainer and arrange for an immediate training session. Any selected trainer profile may be passed to the exercise buddy map-router 304 to obtain directions from the exercise buddy's current location. The exercise buddy trainer profile searcher stores prior searches and related operating data for use when operating into a local exercise buddy datastore 310 as needed.

The exercise buddy messenger 306 permits the exercise buddy to send and receive messages with one or more available trainers. The exercise buddy messenger 306 generates and transmits messages to a trainer mobile application 101 once the exercise buddy and trainer have connected via an exercise buddy query. The exercise buddy messenger 307 receives responses to the exercise buddy's messages from the trainer mobile application 101 once the exercise buddy and trainer have connected via an exercise buddy query. The exercise buddy controls the initiation of all interaction to minimize potential exercise buddies from unsolicited messages. The exercise buddy messenger 307 retains all messages sent and received to help the exercise buddy and trainer maintain a relationship over time as desired. The exercise buddy messenger 306 stores all of the messages to and from trainers for later use into a local exercise buddy datastore 310 as needed.

The exercise buddy UI 307 provides input and output processing to provide an exercise buddy with messages and data needed to perform the trainer profile searches, available trainer searches, communications with trainers, and mapping routes to trainers. This exercise buddy UI 307 also accepts commands from the exercise buddy to instruct the application to perform these tasks.

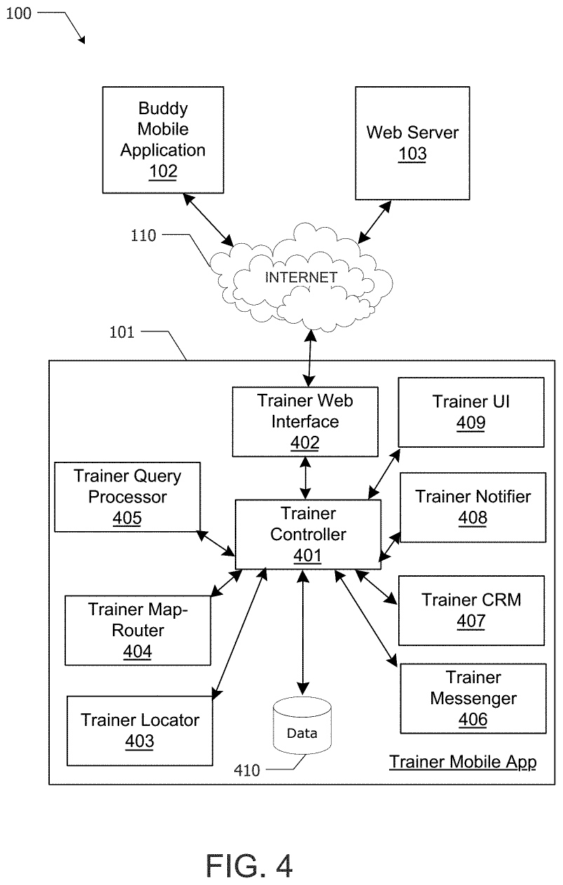

FIG. 4 illustrates another example embodiment of a block diagram of a trainer mobile application within a system for an online, real-time exercise buddy and trainer matching service according to the present invention. The trainer mobile application 101 communicates with the web server 103 and an exercise buddy mobile application 102 over the Internet 110.

The trainer mobile application 101 comprises a set of processing components to perform the processing described herein. The trainer set of processing components includes a trainer controller 401, a trainer web interface 402, a trainer locator 403, a trainer map router 404, a trainer query processor 405, a trainer's profile searcher 306, a trainer messenger 406, a trainer customer relationship manager (CRM) 407, a trainer notifier 408, and a trainer UI 409.

The trainer controller 401 receives available trainer queries via the trainer web interface 402 when it arrives from the mobile application 101. The trainer controller 401 also obtains current GPS location data from the trainer locator 403 for use in providing location data to the web server 103 and the exercise buddy mobile application 102. The trainer controller 401 interacts with the remaining trainer set of processing components to generate messages and data sent to the exercise buddy mobile application 102 and the web server 103 as needed. The trainer controller 401 stores its data for use when operating into a local trainer datastore 410 as needed.

The trainer web interface 402 connects the mobile application 101 to the Internet 110 to send and receive communication from one or more exercise buddy mobile applications 102 and the web server 103. The trainer web interface 402 performs all necessary data formatting, data packet creation, data encryption for security, and data transmission and reception when the trainer mobile application 101 communicates with other processing systems disclosed herein. The trainer web interface 402 also is responsible for ensuring reception of any communications to other computing systems and for logging any errors or attempts to hack into its local data store 410.

The trainer locator 403 obtains current GPS location data from a GPS receiver present in the mobile devices. The trainer locator 403 also provides the trainer's current location periodically to the web server 103 for use in generating and maintaining locations and identities of available trainers for exercise buddies to locate. The trainer locator 403 may maintain a last known location for use when current location data is unavailable. The trainer locator 403 also may determine a current location using Wifi hot spot data and smartphone cell tower data when GPS data is unavailable.

The trainer map router 404 generates a route to an exercise buddy's location or a meeting location from the trainer's current location. The trainer map router 404 obtains current GPS location data from a GPS receiver present in the mobile devices and also may determine the trainer's current location using Wifi hot spot data and smartphone cell tower data when GPS data is unavailable. The trainer map router 404 may use map and location data from the web server 103 or other online map and route travel service such as Apple Maps™, Google Maps™, and similar web services that provide APIs for use by applications on mobile devices. A route generated by the trainer map router 404 may be optimized to generate a route having the lowest travel time, the lowest tolls, and the lowest amount of congestion from accidents, construction and the like. The trainer map router 304 stores saved addresses and routes on available trainers and related operating data for use when operating into a local trainer datastore 310 as needed.

The trainer query processor 405 receives an exercise buddy query requesting an available trainer as identified in data obtained from a trainer search and presents them the exercise buddies via the trainer 409. The trainer query processor 405 obtains input from the trainer whether to accept the query and generates a response to the exercise buddy query that it then transmits to the exercise buddy mobile application 102. The exercise buddy query generator 305 receives all responses to these exercise buddy queries and presents the exercise buddies via the exercise buddy UI 307.

The trainer messenger 406 permits the trainer to receive and respond to messages with an interested exercise buddy. The trainer messenger 406 generates and transmits messages to an exercise buddy mobile application 102 once the exercise buddy and trainer have connected via an exercise buddy query. The trainer messenger 406 receives responses to the trainer's messages from the trainer mobile application 101 once the exercise buddy and trainer have connected via an exercise buddy query. The exercise buddy controls the initiation of all interaction to minimize potential exercise buddies from unsolicited messages. The trainer messenger 406 retains all messages sent and received to help the exercise buddy and trainer maintain a relationship over time as desired. The trainer messenger 406 stores all of the messages to and from trainers for later use into a local exercise buddy datastore 410 as needed.

The trainer customer relations manager (CRM) 407 allows the trainer to organize and retain interested exercise buddy information that is useful in maintaining an ongoing business relationship. The trainer CRM 307 may retain all exercise buddy queries and corresponding responses, and any follow-up interactions to permit a trainer to be able to review the efforts made to assist each particular exercise buddy. The trainer CRM 407 consolidates all of the trainer's business activities within the trainer mobile application 101 that the trainer routinely uses to inform exercise buddies of their availability and current location. As such, the trainer may search and respond to an exercise buddy whenever they are able to use their mobile device. The trainer CRM 407 stores all exercise buddy interactions and message data with exercise buddies into a local exercise buddy datastore 410 as needed.

The trainer notifier 408 generates notifications to the trainer when exercise buddy queries and messages are received. Because the trainer identifies when he/she is available, the trainer may be involved in other activities while awaiting a query. The receipt of an exercise buddy query when they have announced and identified themselves to be available should generate a timely, if not immediate response to the exercise buddy. The trainer notifier 408 activates any notification mechanism supported by the mobile device. The notifications may include alert and alarm sounds, perceptible vibration, and visual indications that the mobile application 101 is seeking the trainer's attention. The trainer may set preferences in the trainer notifier 408 that are associated with the particular alert and alarm sounds, the sound volume, the nature and length of the vibrations, and the level of intrusiveness desired by the trainer. The trainer notifier 408 stores all notification preferences and a log of all exercise buddy queries into a local exercise buddy datastore 410 as needed.

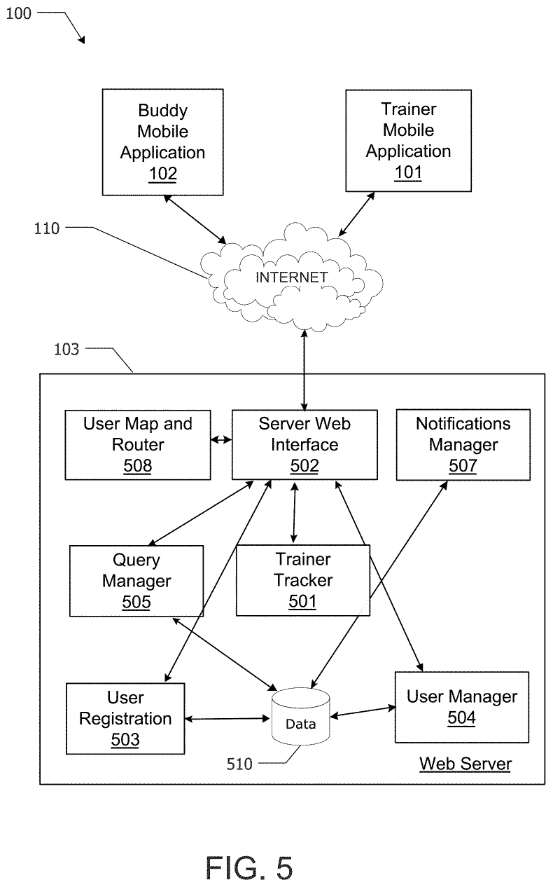

FIG. 5 illustrates another example embodiment of a block diagram of an exercise buddy's mobile application within a system for providing an online, real-time exercise buddy and trainer matching service according to the present invention. The web server 103 communicates with the trainer mobile application 101 and an exercise buddy mobile application 102 over the Internet 110. The web server 103 comprises a set of processing components to perform the processing described herein. The web server set of processing components includes a trainer tracker 501, a server web interface 502, a user registration processor 503, a user manager 504, a query manager 505, an offer manager 506, a notification manager 507, and a user map and router 508.

The trainer tracker 501 communications with a trainer mobile application 101 to maintain location information for a trainer when logged into the system and indicating that he/she are available to meet with exercise buddies. The trainer mobile application 101 communicates with the trainer tracker 501 to set a status of offline, available, and busy. The trainer tracker 501 provides exercise buddies mobile application 102 with locations of nearby available trainers in response to a request from the exercise buddies. The trainer tracker 501 works with the user map and router 508 to provide travel routes for users to meet each other at an agreed upon location.

The server web interface 502 connects the web server 103 to the Internet 110 to send and receive communications from one or more exercise buddy mobile applications 102 and one or more trainer mobile applications 101. The trainer web interface 402 performs all necessary data formatting, data packet creation, data encryption for security, and data transmission and reception when the trainer mobile application 101 communicates with other processing systems disclosed herein. The trainer web interface 402 also is responsible for ensuring reception of any communications to other computing systems and for logging any errors or attempts to hack into the its local data store 410.

The user registration processor 503 receives and processes requests to create user accounts for both exercise buddies and trainers from their respective mobile applications. The user registration processor 503 accepts required data from each new user and populates profile information into the datastore for use when matching and communicating with parties using the mobile applications. The user registration processor 503 works with the user manager 504 that performs the online interaction with users interacting with the web server 103.

The user manager 504 processes user log in attempts to authenticate the users via their respective mobile applications. The user manager 504 may utilize biometric data verification in the mobile applications as a form of authentication. The user manager 504 may also use usernames and passwords as well as multi-factor authentication to authenticate users before permitting them to process requests with the web server 103.

The query manager 505 connects an exercise buddy's query with a requested nearby trainer via their respective mobile applications. All communications between the mobile applications passes through the server for monitoring, logging, and status monitoring. The query manager 505 receives an exercise buddy's query, passes the query to the trainer mobile application 101, and receives a response that is then forwarded to the exercise buddy's mobile application 102.

He offer manager 506 receives, maintains, forwards, and stores communication between buddies and trainers associated with offers from trainers and acceptance from buddies for training services. A contract for services in electronic form is created and exchanged by the parties containing price and services provided terms defined. The contract with executed copies of the agreement is maintained by the offer manager 506 for retrieval as needed.

The notification manager 507 provides all system notifications to exercise buddies' mobile applications 102 and trainers' mobile applications 101 for appointments, messages, and offers as needed.

The user map and router 508 generates travel routes for exercise buddy's and trainers to travel from a current location to a meeting location. The user map and router 508 provides maps of the local areas to all users and the maps used by the trainer tracker 501 when communicating available trainer location data to exercise buddies mobile application 102.

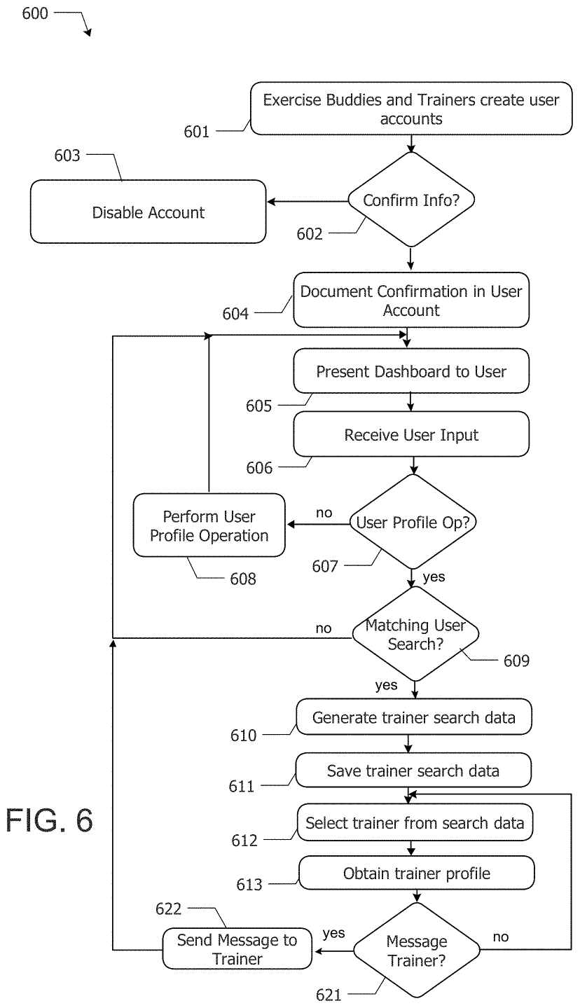

FIG. 6 illustrates a flowchart corresponding to a method performed by software components providing a system for an online, real-time exercise buddy and trainer matching service according to the present invention. The process 600 begins with exercise buddies and trainers creating a user account in step 601. Step 602 attempts to confirm the details of the user account by contacting the user using one or more of the provided contact method such as email, telephone voice call or mobile SMS text. A message may be sent using one or more of the provided communication mechanisms that contains a unique code which is valid for a short period of time. The user is expected to provide the unique code to the system to confirm the user may be contacted at the provided address or number. If the confirmation is not successful, step 603 disables the user account, preventing the user from interacting with the web server 103 and system 100.

If the confirmation is successful, step 604 documents the confirmation and enables the user account for use with a trainer mobile application 101 or an exercise buddy mobile application 102. Step 605 presents a dashboard of operations that may be performed. Based upon the user's input received in step 606, the user may choose the user profile operation. Test step 607 determines whether the user input corresponds to a user profile operation, and if so, the user profile operation allows a user to create, view, modify, and delete the user profile in step 608. Once an operation is completed the mobile applications 101-102 returns to the dashboard until the user logs out.

When test step 607 determines whether the user input does not corresponds to a user profile operation, test step 609 determines whether the user input corresponds to a search for trainer operation, and if not, the process returns to the dashboard in step 606; otherwise in step 610, the exercise buddy application 102 generates a search for available trainers near the exercise buddy's location. The search results generated are presented to the exercise buddy for consideration.

The user may save the search results in step 611. In step 612, the mobile application requests the trainer's profile. The exercise buddy may select and preview available trainers in the local area in step 613. The profile data is obtained in step 613 from the web server 103 from a trainer database that is part of the web server.

Using the above process 600, the exercise buddy can search other areas and locate a local trainer through the application 102. The exercise buddy also may save one or more trainer profiles found in the search results into a set of favorites for later retrieval.

When an exercise buddy decides to contact a trainer based upon a profile, the exercise buddy chooses the send a message operation. Test step 621 determines whether the user wants to send a message to the selected trainer, and if so, step 622 permits the user to compose and sent a message requesting a connection; otherwise the process 600 returns to step 612 review additional trainer profiles.

When sending a message to a trainer the mobile application 102 contacts the web server 103 to submit a request in the form of an electronic message. A message is sent to notify the trainer's mobile application 101 for the trainer who posted a profile that a request has been made. The user may search for and contact as many trainers located in multiple locations as desired.

The embodiments described herein are implemented as logical operations performed by a computer. The logical operations of these various embodiments of the present invention are implemented (1) as a sequence of computer-implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise. Any singular term used in this present patent application is applicable to its plural form even if the singular form of any term is used.

In the present application, all or any part of the invention's software or application(s) or smart device application(s) may be installed on any of the user's or operator's smart device(s), any server(s) or computer system(s) or web application(s) required to allow communication and transfer of content(s) or data between any combination of the components.

What is claimed:

1. A system for providing an online, real-time exercise buddy and trainer matching service, the system for interconnecting a trainer mobile device, one or more exercise buddy mobile devices, and a web server over the Internet, the system comprising:
the trainer mobile device comprises:
a trainer tracker for maintaining location information for a trainer when logged into the system and indicating that he/she are available to meet with exercise buddies;
a user manager for authenticating users via utilizing biometric data verification in the mobile applications as a form of authentication and multi-factor authentication;
a query manager for connects an exercise buddy's query with a requested nearby trainer;
an offer manager for receiving, maintaining, forwarding, and storing communication between buddies and trainers associated with offers from trainers and acceptance from buddies for training services;
a notification manager 507 for providing notifications to exercise buddies and trainers for appointments, messages, and offers; and
a user map and router for generating travel routes for exercise buddies and trainers to travel from a current location to a meeting location.

2. The system according to claim 1, wherein the trainer mobile device and the buddy mobile device comprise smartphones, tablets, and laptop computers.

3. The system according to claim 2, wherein the web server may be utilized for queries, offers, meeting scheduling, and user functions via a web browser.

4. The system according to claim 1, wherein the user map and router obtains GPS location data from the mobile devices using built-in GPS receivers.

5. The system according to claim 1, wherein the system utilizes in-app currency provided in in-app coins.

6. The system according to claim 5, wherein the in-app coins may be traded by users.

7. The system according to claim 6, wherein the in-app coins can be redeemed for cash each month.

8. The system according to claim 1, wherein the trainer and training buddy may meet for training virtually using video-based communication.

9. The system according to claim 8, wherein the query manager further performs searching for trainers having desired characteristics and services.

10. The system according to claim 9, wherein the query manager shares training buddy queries with nearby trainers and generating trainer offers to training buddies.

11. A method for providing an online, real-time exercise buddy and trainer matching service, the system for interconnecting a trainer mobile device, one or more exercise buddy mobile devices, and a web server over the Internet, the method comprising:
defining trainer characteristics and services to be obtained;
searching for available trainers matching the trainer characteristics and services;
selecting a first available trainer to solicit an offer;
transmitting a message to the first available trainer;
receiving an offer for services; and
accepting the offer for services.

12. The method according to claim 11, wherein the offer for services is transmitted by the first available trainer.

13. The method according to claim 11, wherein the offer for services is transmitted by a second available trainer before receiving a message from the training buddy.

14. The method according to claim 11, wherein the web server utilizes in-app currency provided in in-app coins.

15. The method according to claim 14, wherein the in-app coins may be traded by users.

16. The method according to claim 15, wherein the in-app coins can be redeemed for cash each month.

17. The method according to claim 11, wherein the trainer and training buddy may meet for training virtually using video-based communication.

18. The method according to claim 11, wherein the trainer mobile device and the buddy mobile device comprise smartphones, tablets, and laptop computers.

19. The method according to claim 18, wherein the web server may be utilized for queries, offers, meeting scheduling, and user functions via a web browser.

20. The method according to claim 11, wherein the method further:
obtains location data for available trainers and training buddies;
utilizes the location data in searching; and
generates a travel route presented with a map for the training buddies and trainers.
